(19) United States
(12) Patent Application Publication
(10) Pub. No.: US 2012/0296370 A1
(43) Pub. Date: Nov. 22, 2012

(54) INJECTION PRESSURIZER

(75) Inventor: Erik Hansson, Uppsala (SE)

(73) Assignee: ST. JUDE MEDICAL SYSTEMS AB, Uppsala (SE)

(21) Appl. No.: 13/522,657

(22) PCT Filed: Jan. 19, 2011
(86) PCT No.: PCT/SE2011/050057

§ 371 Date: Jul. 17, 2012

Related U.S. Application Data
(60) Provisional application No. 61/296,181, filed on Jan. 19, 2010.

(30) Foreign Application Priority Data
Jan. 19, 2010 (SE) 1050058-5

(51) Int. Cl. A61B 17/135

(52) U.S. Cl. 606/202

(57) ABSTRACT

The invention relates to an injection pressurizer for pressurizing an inflatable device comprising:—a housing enclosing a first cavity;—a plunger adapted to fit inside said housing and provided with a plunger sealing unit adapted to define a second cavity inside said housing, the plunger is further provided with an end part at the end opposite the plunger sealing unit; wherein the plunger further comprises
an elongated tube defining a tube cavity extending from said plunger sealing unit to said end part of the plunger, said tube cavity being in fluid communication with said second cavity via an opening in said plunger sealing unit; and a pressure indicating piston adapted to be moveably arranged inside said tube cavity, such that the piston delimits a first volume (A) in said tube cavity from a second volume (B), such that when a pressure is built up in the second volume (B), the piston is moved by the pressure to equalize the pressures of said first volume (A) and said second volume (B), and thereby indicates the pressure in the second volume (B). The invention relates according to another aspect to a method of pressurizing an inflatable device with an injection pressurizer.

### FIELD OF THE INVENTION

The present invention relates to an injection pressurizer for pressurizing an inflatable device and a method in connection thereto. In particular, the invention relates to an injection pressurizer for pressurizing an inflatable device adapted to be used against a puncture site in a vessel of a patient to achieve haemostasis.

### BACKGROUND OF THE INVENTION

To access a patient's vascular system for an invasive medical procedure such as catheterization or similar procedures, a puncture is made in e.g. the femoral artery or the radial artery. Following the medical procedure the flow of blood through the puncture site has to be stopped, so that haemostasis may begin as soon as possible. This may be done by using a compression device.

One example of such a compression device is known from EP 1382306, disclosing a haemostatic device with an inflatable balloon. A main balloon is pressed against a puncture site of a patient and filled with a first fluid to inflate the balloon. A pressing member is further provided on the main balloon, and when filled with a second fluid, it presses against the main balloon to apply a compressive force to the puncture site that acts in an inclined direction with respect to the surface of the limb of the patient. The main balloon and the pressing member are held in place against the puncture site with a flexible band and a curved plate. Inflation of the balloon is carried out by inserting the protruding tip of a syringe into a connector to the balloon and pushing a plunger on the syringe to introduce fluid within the syringe into the balloon. Once fluid has been injected into the balloon and the protruding tip of the syringe is withdrawn from the connector, a check valve closes preventing the fluid from leaking out and thus maintaining the balloon in an inflated state.

Hence, it is only the injected fluid volume from the syringe that is taken into respect when applying a pressure to the puncture site. If, for example, the flexible belt is tightened hard against the body, this tightening will contribute to the compression on the puncture site, as the balloon then already when not inflated may exert some pressure on the puncture site. If only taking the injected fluid volume in respect, the actual pressure on the puncture site may be erroneously reflected, and it may be difficult to correctly adjust the pressure afterwards.

It is an object of the invention to provide an improved device that enables an accurately applied pressurization against a puncture site, and in particular enables a following adjustment of the applied pressure to the puncture site.

It is a further object of the invention to provide an improved device which is easy to handle and economical to manufacture and ship to the end user.

### SUMMARY OF THE INVENTION

The above-mentioned objects are achieved by an injection pressurizer for pressurizing an inflatable device comprising: a housing enclosing a first cavity; a plunger adapted to fit inside said housing and provided with a plunger sealing unit adapted to define a second cavity inside said housing, the plunger is further provided with an end part at the end opposite the plunger sealing unit. The plunger further comprises: an elongated tube defining a tube cavity extending from the plunger sealing unit to the end part of the plunger, the tube cavity being in fluid communication with the second cavity via an opening in the plunger sealing unit; and a pressure indicating piston adapted to be moveably arranged inside said tube cavity, such that the piston delimits a first volume A in the tube cavity from a second volume B, such that when a pressure is built up in the second volume B, the piston is moved by the pressure to equalize the pressures of the first volume A and the second volume B, and thereby indicates the pressure in the second volume B.

Thus, the invention enables a correct application of pressure at the puncture site, as the real total pressure on the puncture site is indicated by the pressure indicating piston. Further, the invention enables a correct adjustment of the pressure applied to the puncture site.

When applying pressure on a puncture site in the vessel to achieve haemostasis, it is important to not totally block the blood flow in the vessel. Therefore the pressure on the puncture site is relieved after a while to be sure not to stop the blood flow totally. When using a pressurizer according to the invention, the blood pressure in the vessel may be directly compared to the indicated pressure of the pressurizer, and adjusted such that a desired pressure in the vessel is achieved. The pressure against the puncture site on the vessel may thus be regulated with respect to the blood pressure in the vessel.

According to another aspect, the injection pressurizer comprises a plunger cap to enable calibration of the injection pressurizer to the air-pressure in the environment in which the pressurizer should be used. Thus, the function of the injection pressurizer is not compromised by the surrounding air pressure.

According to a further aspect, the injection pressurizer comprises a locking mechanism to enable locking of the injection pressurizer at a desired obtained pressure. Thus, a user of the pressurizer does not have to maintain a desired obtained pressure by manpower.

The injection pressurizer is according to one embodiment a disposable article preferably made of plastic material. The need for sterilization of the pressurizer is then obviated, and the pressurizer is thus easier to handle than prior art devices that have to be sterilized. The pressurizer may be moulded and is thus easy to manufacture. As the pressurizer is adapted to indicate the applied pressure, the need for extra measurement equipment is omitted. The provided pressurizer is thus a compact device which is easy to use and ship to the end user. If the pressurizer is made of a light-weight material such as plastic, the weight of the pressurizer will be low which will further facilitate shipping to the end user.

The invention relates according to another aspect to a method for pressurizing an inflatable device with an injection pressurizer. Thus, a method of pressurizing an inflatable device is shown which enables a correct reflection of the applied pressure.

Preferred embodiments are set forth in the dependent claims and in the detailed description.

### SHORT DESCRIPTION OF THE APPENDED DRAWINGS

Below the invention will be described with reference to the appended figures, of which:

FIG. 1 shows an example of an injection pressurizer according to the invention in an unassembled state.

FIG. 2 shows an example of an injection pressurizer according to the invention in an assembled state.

FIG. 3 shows a cross-section along A-A of the injection pressurizer in FIG. 10.

FIG. 4 illustrates an example of an injection pressurizer according to the invention when the plunger cap is closed.

FIG. 5 illustrates an example of an injection pressurizer according to the invention when the plunger cap is open.

FIG. 6 illustrates an embodiment of a housing according to the invention when seen from below.

FIG. 7 shows a cross-section along B-B of the housing in FIG. 6.

FIG. 8 shows a cross-section along C-C of the housing in FIG. 7.

FIG. 9 shows an enlarged view of a detail of FIG. 8.

FIG. 10 illustrates an injection pressurizer with a locking mechanism according to one embodiment of the invention.

FIG. 11 illustrates an enlarged view of the locking mechanism illustrated in FIG. 10. FIG. 12 illustrates an injection pressurizer with a locking mechanism according to another embodiment of the invention.

FIG. 13 illustrates an enlarged view of the locking mechanism illustrated in FIG. 12.

FIG. 14 illustrates an injection pressurizer with a locking mechanism according to one embodiment of the invention, when the locking mechanism is in its locked state.

FIG. 15 shows a cross-section along D-D of FIG. 14.

FIG. 16 illustrates an injection pressurizer with a locking mechanism according to one embodiment of the invention, when the locking mechanism is in its unlocked state.

FIG. 17 shows a cross-section along E-E of FIG. 16.

### DETAILED DESCRIPTION OF PREFERRED EMBODIMENTS OF THE INVENTION

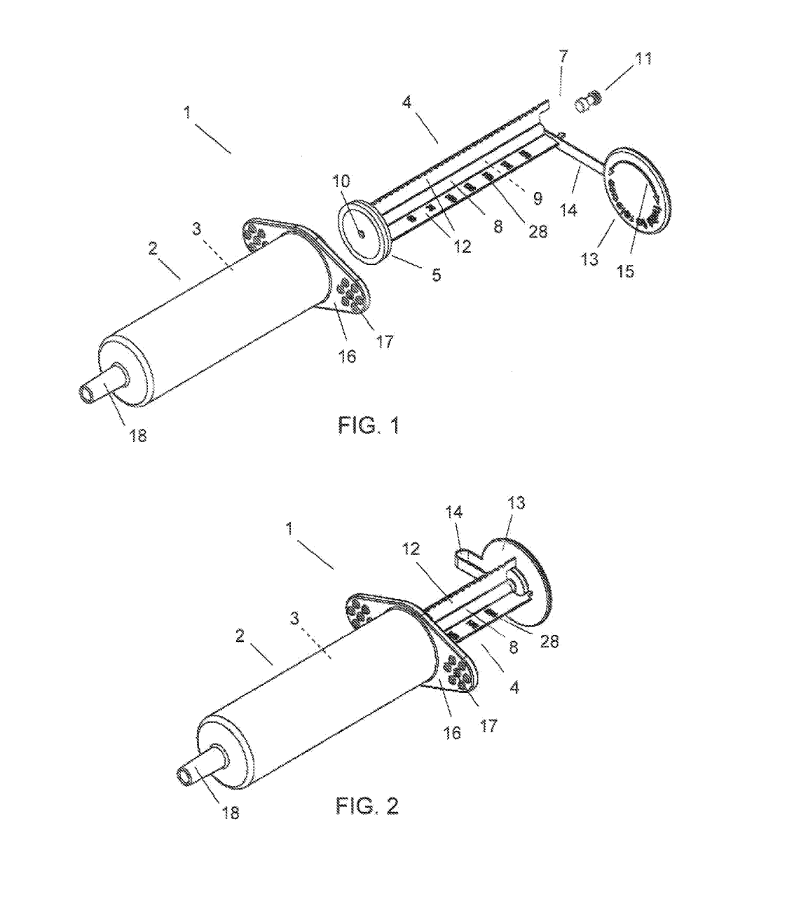

In FIG. 1 an injection pressurizer 1 according to the invention for pressurizing e.g. an inflatable device to achieve haemostasis is shown. The pressuizer 1 may for example be applied in connection with a femoral compression device as disclosed in WO 2009/000665 to inflate the therein described inflatable air cushion, or in connection with a radial compression device as disclosed in WO 96/25110, with the difference that the compression element in WO 96/25110 then is provided with an inflatable device.

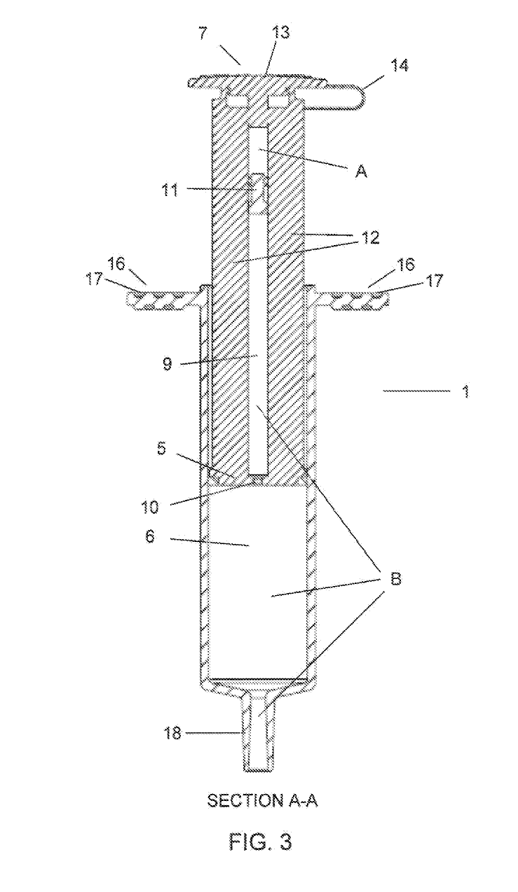

The pressurizer 1 in FIG. 1 comprises a housing 2 enclosing a first cavity 3, and a plunger 4 adapted to fit inside the housing 2, which is provided with a plunger sealing unit 5 adapted to define a second cavity 6 (FIG. 3) inside the housing 2. The plunger 4 is further provided with an end part 7 at the end opposite the plunger sealing unit 5. The plunger 4 also comprises an elongated tube 8 defining a tube cavity 9 extending from the plunger sealing unit 5 to the end part 7 of the plunger 4. The tube cavity 9 is in fluid communication with the second cavity 6 via an opening 10 in the plunger sealing unit 5. The plunger 4 also comprises a pressure indicating piston 11 adapted to be moveably arranged inside said tube cavity 9, such that the piston 11 delimits a first volume A in said tube cavity 9 from a second volume B, such that when a pressure is built up in the second volume B, the piston 11 is moved by the pressure to equalize the pressures of said first volume A and said second volume B, and thereby indicates the pressure in the second volume B (FIG. 3). The pressurizer 1 may according to one embodiment also comprise a plunger cap 13 with an attachment 14 to the plunger 4, and preferably provided with an instruction on its upper side to e.g. instruct a user how to use the pressurizer 1.

According to one embodiment, the plunger 4 comprises a scale 28 in its lengthwise direction adapted to indicate pressure in the second volume B in conjunction with said piston 11. Thus, by having a scale 28 on the plunger 4, and preferably a scale indicating pressure, a pressure applied by the pressurizer 1 may be measured and instantly read off the same. The injection pressurizer 1 is according to one embodiment adapted to pressurize an inflatable device in the area of 0 to 300 mmHg. The scale 28 is then advantageously indicating the pressure in the area 0 to 300 mmHg.

The tube 8 is preferably located essentially in the centre of the plunger 4 as illustrated in the figures. To achieve such a centralization, the plunger 4 is according to one embodiment provided with flanges 12 attached to the tube 8 for maintaining the tube 8 centrally in the housing 2, and to support the construction. According to one embodiment, the plunger 4 is provided with four flanges 12, but any number of flanges is possible as long as the flanges 12 achieve centralization of the tube 8 in the plunger 4. Each of the flanges 12 extends from the outside of the tube 8 almost to the inner surface of the housing 2, when the plunger 4 is fitted inside the housing 2. According to one embodiment, the scale 28 is provided on one or several of the flanges 12.

The housing 2 may also be provided with grips 16 to make it easier to hold and control the pressurizer 1. The grips 16 may be provided with depressions 17, elevations 17 or other kinds of anti-slip means 17. The housing 2 is preferably also provided with an extended nozzle 18 adapted to be in fluid communication with the second cavity 3. The nozzle 18 is preferably adapted to be connected to e.g. a fluid connection adapted to be in fluid connection with an inflatable device for inflation of the same.

FIG. 2 is illustrating the pressurizer 1 in its assembled state, and the pressure indicating piston 11 is inside the tube 8 in the tube cavity 9.

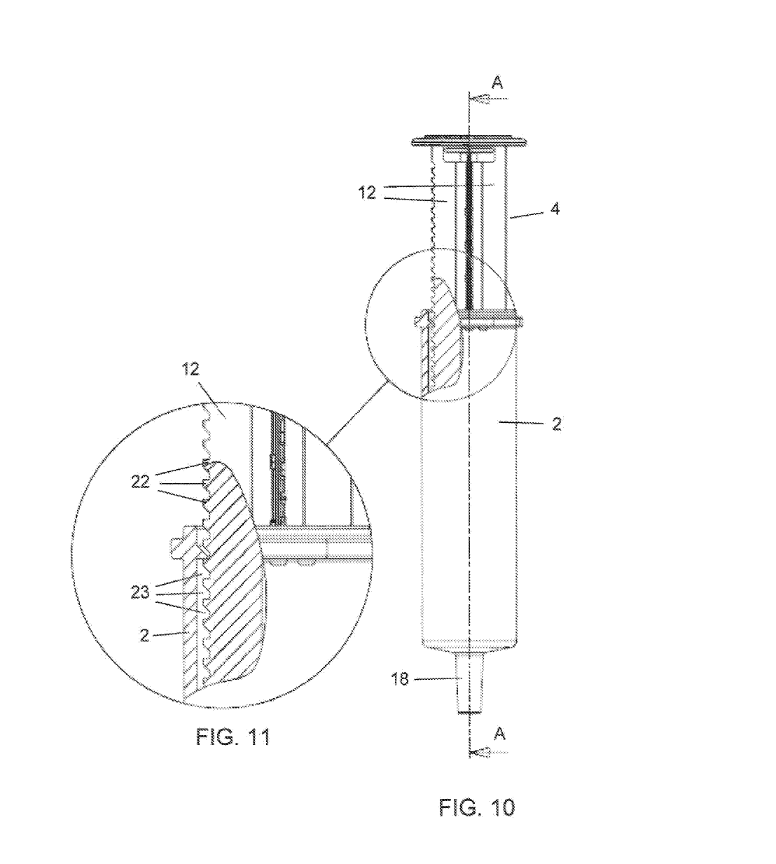

The function of the pressurizer 1 will now be explained with reference to FIG. 3. FIG. 3 is showing a cross-section of the pressurizer 1 in FIG. 10 along A-A. Before the pressurizer 1 is pressurized, the pressure indicating piston 11 is located inside and at the bottom of the tube cavity 9, next to the plunger sealing unit 5. Thus, the pressure in second volume B now equals the pressure of ambient air. The pressure indicating piston 11 is as explained adapted to be moveably arranged inside the tube cavity 9. The piston 11 is matched to the wall of the tube cavity 9 to not allow any fluid from passing between the wall of the tube cavity 9 and the piston 11, thus, the piston 11 fit snuggly against the wall of the tube cavity 9. A first volume A is defined as the volume in the tube cavity 9 delimited by the inner side of the tube cavity 9, the piston 11 and the end part 7 of the plunger 4 including in some embodiments the plunger cap 13. A second volume B is defined as the volume in the tube cavity 9 delimited by the inner side of the tube cavity 9, the piston 11 and the plunger sealing unit 5, plus the volume of the opening 10 in the plunger sealing unit 5, plus the volume defined by the plunger sealing unit 5, the inner side of the second cavity 6 inside the housing 2 and the inner side of the nozzle 18, plus the volume in the inflatable device which the pressurizer 1 intends to inflate and when existent, the volume in a fluid connection between the nozzle 18 and an inflatable device.

The fluid used in connection with the pressurizer 1 may be air or another suitable gas or liquid. If the fluid is not air, the pressurizer 1 is delivered to the end user with a sealed volume A enclosing the fluid.

A fluid connection and an inflatable device attached to the nozzle are omitted in FIG. 3. The pressurizer 1 in FIG. 3 is pressurized by engaging and pressing the plunger 4 into the housing 2, thus making the volume of the second cavity 6 smaller. When a pressure is built up in the second volume B, the piston 11 is moved by the pressure to equalize the pressures of the first volume A and the second volume B, and thereby indicates the pressure in the second volume B on the scale 28. The pressure of the first volume A and the second volume B are thus the same. The pressure of the second volume B, which is the real pressure that is applied to e.g. a puncture site of a patient when the pressurizer 1 is used for achieving haemostasis, may then easily be observed and adjusted if needed. To achieve a correct measurement of the pressure, it is important that the pressurizer 1 is connected to e.g. an inflatable device before the inflatable device is applied to the puncture site in a vessel in a body of a person or similar.

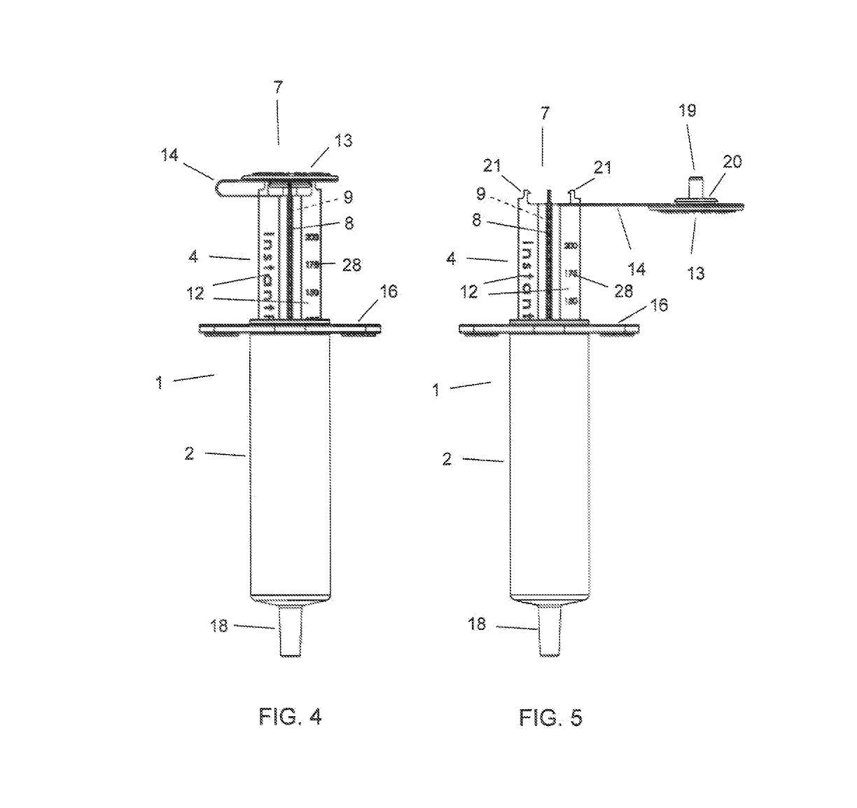

According to one embodiment, and illustrated in FIGS. 4 and 5, the end part 7 comprises a plunger cap 13 which is adapted to seal the first volume A. Preferably, the plunger cap 13 is detachably arranged 19, 20, 21 on the plunger 4. By having a plunger cap 13 to seal the first volume A, it is possible to calibrate the pressurizer 1 to the surrounding air pressure where the pressurizer is used. FIG. 4 illustrates the pressurizer 1 when the plunger cap 13 is closed. FIG. 5 illustrates the pressurizer 1 when the plunger cap 13 is open. According to one embodiment, the plunger cap 13 is provided with a pin 19 to seal the tube cavity 9 in the elongated tube 8. The pin 19 is then adapted to match an opening in the elongated tube 8. To enable a correct positioning of the pin 19 on the elongated tube 8, the plunger 4 is according to one embodiment provided with supporting means 21 to support an extension 20 provided on the plunger cap 13. When the plunger cap 13 is closed as illustrated in FIG. 4, the extension 20 mates with the supporting means 21 and thereby it is secured that the pin 19 is correctly positioned over the opening in the elongated tube 8 to seal volume A. In FIGS. 4 and 5 the supporting means are shown as four extensions extending from the flanges 12, each having an angled upper side to receive the extension 20 on the plunger cap 13. The extension 20 is illustrated as a circular elevation from the plunger cap 13. The embodiments illustrated in the figures should not be seen as limiting the scope of the invention, and other kinds of supporting means 21 and extensions 20 are possible to enable a correct sealing of volume A. For example, the pin 19 could according to another embodiment be adapted to fit directly into the tube cavity 9 to securely seal volume A. Thus, by having a plunger cap 13, the pressurizer 1 may be calibrated and an indication of the pressure is obtained without errors obtained from different ambient pressures.

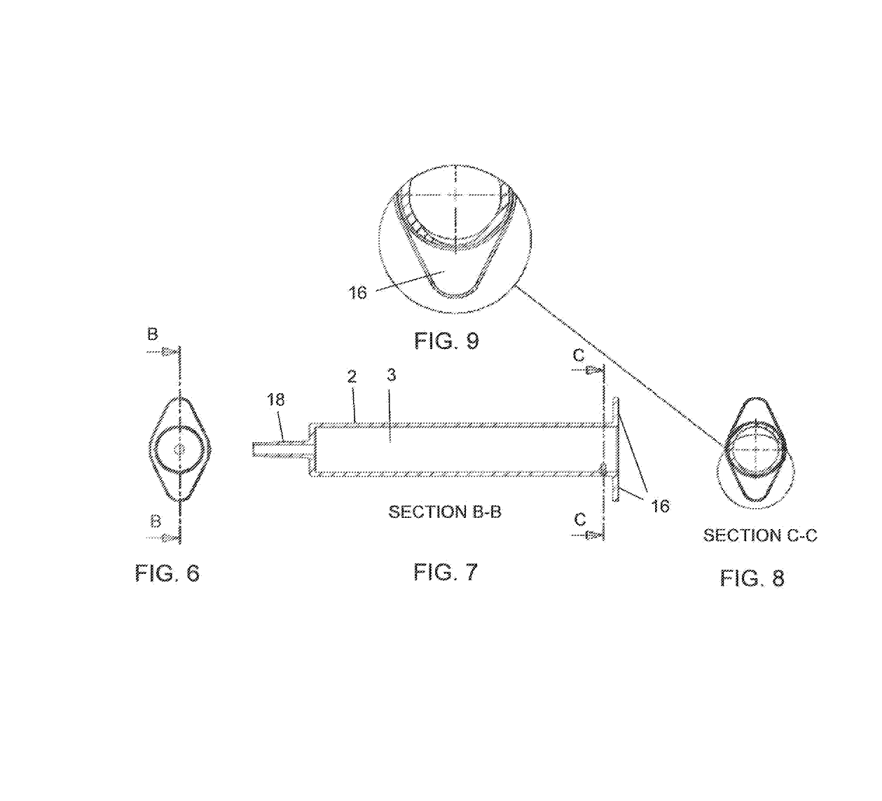

FIG. 6 illustrates a housing 2 according to one embodiment of the invention seen from above, and FIG. 7 shows a cross-section along B-B of the housing 2 in FIG. 6. Here the first cavity 3 in the housing 2 is illustrated, which is delimited by the inner side of the housing 2. The housing 2 preferably has a circular cross-sectional shape, but other shapes such as oval are also possible. FIG. 8 shows a cross-section along C-C of the housing 2 in FIG. 7. FIG. 9 show an enlarged view of a grip 16 attached to the housing 2 to make it easier to hold and control the pressurizer 1.

As illustrated in FIGS. 10-17, the injection pressurizer 1 comprises, according to one embodiment, a locking mechanism 22, 23, 24, 25, 26, 27 adapted to lock the plunger 4 to the housing 2 when e.g. a desired pressure in the second volume B is obtained. A user then does not have to hold the piston 4 in relation to the housing 2 in its pressurized state, instead the user may rely on the locking mechanism 22, 23, 24, 25, 26, 27 to securely hold the pressurizer 1 pressurized.

In FIG. 10 the pressurizer 1 is shown with one embodiment of the locking mechanism 22, 23, which is illustrated in an enlarged view in FIG. 11. The locking mechanism 22, 23 comprising a first engaging means 22 located on at least one flange 12 of the plunger 4 which mates with a second engaging means 23 located on the inner surface of the housing 2. When the first engaging means 22 is engaged with the second engaging means 23, the pressurizer 1 is locked. The first engaging means 22 is located on the side of the flange 12 that faces the inner side of the housing 2. The first engaging means 22 may be provided on one or several flanges 12, and may be cut out as projections 22 in the flange 12. The second engaging means 23 protrudes from the inner side of the housing 2 and is preferably provided along a part of, or the whole of, the length of the inner side of the housing 2, and preferably along a part of the circumference of the inner side of the housing 2.

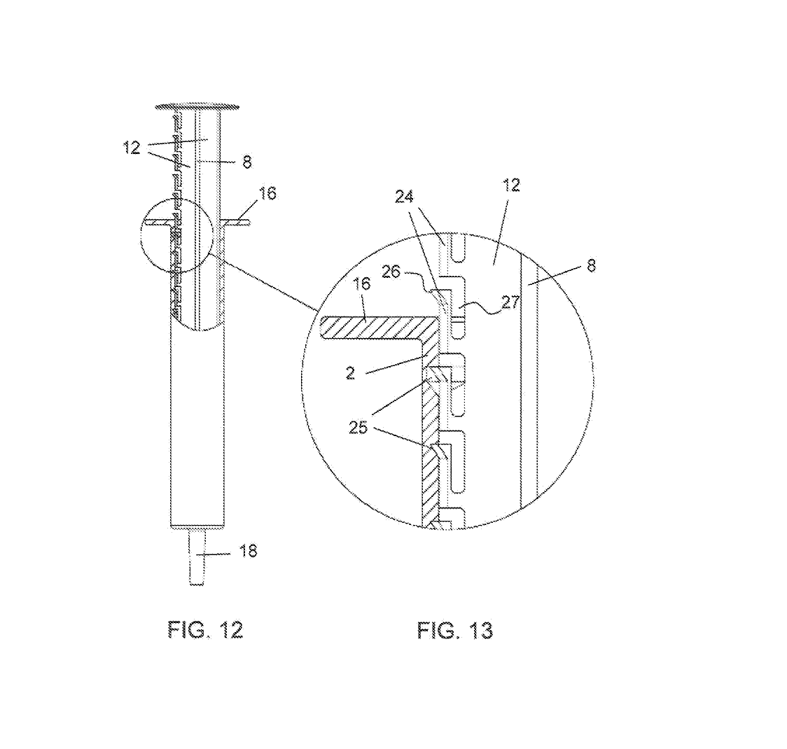

In FIGS. 12 and 13 another embodiment of the locking mechanism 24, 25, 26, 27 is shown. The locking mechanism 24, 25, 26, 27 here comprises a first engaging means 24, 25, 26 located on at least one flange 12 of the plunger 4 which mates with a second engaging means 25 located on the inner surface of the housing 2, and has a similar function to the locking mechanism illustrated in FIGS. 10 and 11. The second engaging means 25 is thus cut out as excavation 25. The first engaging means 24, 26, 27 has a flexible part 24 that is connected to the flange 12 and is adapted to bend into a cavity 27 located between the flexible part 24 and the flange 12. The flexible part 24 further has a small protrusion 26 on its end side that is not connected to the flange 12. When the plunger 4 is engaged and pushed into the housing 2, the flexible part 24 with its protrusion 26 is forced to bend into the cavity when not mating with the excavations 25, and introduction of the plunger 4 into the housing 2 is made possible. When mating, movement of the plunger 4 out of the housing 2 is stopped as the protrusion 26 is acting against this motion as the upper side of the protrusion 26 is mating with one side of the excavation 25 in the direction of the motion. Further movement of the plunger 4 into the housing 2 is thus possible. Thus, a locking mechanism 24, 25, 26, 27 which locks the plunger 4 against redrawing the plunger 4 from the housing 2 is made possible.

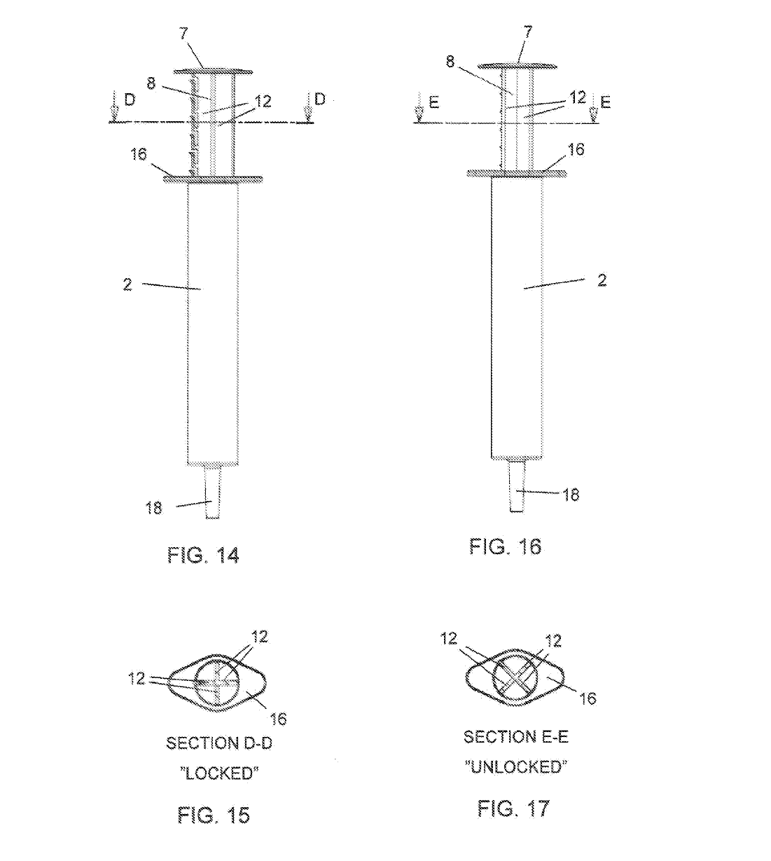

The locking mechanisms 22, 23, 24, 25, 26, 27 illustrated in FIGS. 10 to 13 are adapted, according to one embodiment, to lock respectively release the plunger 4 when the plunger 4 is twisted. There are in this embodiment four flanges 12, but the number of flanges 12 may be more or fewer. The embodiment is illustrated in FIGS. 14 to 17. In FIG. 15 the pressurizer 1 in FIG. 14 is seen from above along D-D. In FIG. 15 the plunger 4 is in its locked state, and this means that withdrawal of the plunger 15 from the housing 2 is not possible. The first engaging means 22, 24, 26, 27 located on at least one flange 12 of the plunger 4 now are engaged with second engaging means 23, 25 located on the inner surface of the housing 2.

In FIG. 17, the pressurizer 1 in FIG. 16 is seen from above along E-E. In FIG. 17 the plunger 4 is in its unlocked state, and this means that withdrawal of the plunger 15 from the housing 2 is possible. The plunger 4 has now been twisted in relation to the housing 2 such that the first engaging means 22, 24, 26, 27 located on at least one flange 12 of the plunger 4 is not engaged with second engaging means 23, 25 located on the inner surface of the housing 2. Thus, the second engaging means 23, 25 is only located on parts of the inner surface of the housing 2, and when the plunger 4 is twisted such that the first engaging means 22, 24, 26, 27 is not opposite any second engaging means 23, 25, the plunger 4 is unlocked. Accordingly, an easily handled locking of the plunger 4 is made possible to maintain a certain pressure of the pressurizer 1.

According to a further embodiment, the injection pressurizer 1 is at least partly made of a transparent material, e.g. glass or plastic. For example, the housing 2 and the tube 8 may be made of a transparent material. Thus, it is possible to see the pressure indicating piston 11 inside the pressurizer 1 and read off the scale 28 independent of how far the plunger 4 is introduced into the housing 2. The figures does not disclose a transparent housing 2, but it may be a necessary feature to be able to read of the scale 28 if the pressure indicating piston 11 is indicating a value on the scale 28 when inside the housing 2.

As mentioned before, the injection pressurizer 1 is according to one embodiment adapted to inflate an inflatable device in a compression system for compressing against a puncture site of a vessel of a patient. The injection pressurizer 1 according to the invention may also be adapted to be used to inflate for example a cuff for blood pressure monitoring, or to inflate a device in another application where an accurate pressure is desired. A further application is inflation and pressurizing of a balloon for expansion and placement of a stent mounted on said balloon in a vessel. By employing the injection pressurizer 1 for this application it is possible to achieve a greater accuracy of the pressure in the balloon.

The invention relates according to another aspect to a method for pressurizing an inflatable device with an injection pressurizer 1. The injection pressurizer 1 comprises a housing 2 enclosing a first cavity 3 and a plunger 4 adapted to fit inside the housing 2. The method comprises: engaging the plunger 4 into the housing 2, attaching the injection pressurizer 1 to the inflatable device, sealing a first volume A in a tube cavity 9 in an elongated tube 8 provided in the plunger 4; pressing the plunger 4 into the housing 2 such that a pressure indicating piston 11 adapted to be moveably arranged inside the tube cavity 9 and delimiting the first volume A in said tube cavity 9 from a second volume B, indicates a desired pressure built up in the second volume B when the piston 11 is moved by the pressure to equalize the pressures of the first volume A and the second volume B. Thus, a method of pressurizing an inflatable device is shown which enables an accurate measurement and display of the applied pressure.

According to one embodiment, the method comprises twisting the plunger 4 to lock the plunger 4 to the housing 2. According to a further embodiment, the method comprises twisting the plunger 4 to unlock the plunger 4 from the housing 2. Thus, a method of maintaining a desired applied pressure is achieved without using manpower.

The present invention is not limited to the above-described preferred embodiments. Various alternatives, modifications and equivalents may be used. Therefore, the above embodiments should not be taken as limiting the scope of the invention, which is defined by the appending claims.

1. Injection pressurizer for pressurizing an inflatable device comprising:
a housing enclosing a first cavity;
a plunger adapted to fit inside said housing and provided with a plunger sealing unit adapted to define a second cavity inside said housing, the plunger is further provided with an end part at the end opposite the plunger sealing unit;
characterized in that said plunger further comprises
an elongated tube defining a tube cavity extending from said plunger sealing unit to said end part of the plunger, said tube cavity being in fluid communication with said second cavity via an opening in said plunger sealing unit;
a pressure indicating piston adapted to be moveably arranged inside said tube cavity, such that the piston delimits a first volume in said tube cavity from a second volume , such that when a pressure is built up in the second volume, the piston is moved by the pressure to equalize the pressures of said first volume and said second volume, and thereby indicates the pressure in the second volume.

2. Injection pressurizer according to claim 1, wherein said plunger comprises a scale in its lengthwise direction adapted to indicate pressure in the second volume in conjunction with said piston.

3. Injection pressurizer according to claim 1, wherein said end part comprises a plunger cap which is adapted to seal said first volume.

4. Injection pressurizer according to claim 3, wherein said plunger cap is detachably arranged on said plunger.

5. Injection pressurizer according to claim 1, wherein said tube is located essentially in the centre of said plunger.

6. Injection pressurizer according to claim 5, wherein said plunger is provided with flanges attached to the tube for maintaining the tube centrally in said housing.

7. Injection pressurizer according to claim 1, comprising a locking mechanism adapted to lock the plunger to the housing when e.g. a desired pressure in the second volume is obtained.

8. Injection pressurizer according to claim 7, wherein said locking mechanism comprises first engaging means located on at least one flange of the plunger which mate with second engaging means located on the inner surface of the housing.

9. Injection pressurizer according to claim 7, wherein said locking mechanism is adapted to lock respectively release the plunger when the plunger is twisted.

10. Injection pressurizer according to claim 1, wherein the injection pressurizer at least partly is made of a transparent material.

11. Injection pressurizer according to claim 1, wherein the housing is provided with an extended nozzle adapted to be in fluid communication with said second cavity.

12. Injection pressurizer according to claim 1, adapted to inflate an inflatable device in a compression system for compressing against a puncture site of a vessel of a patient.

13. Method for pressurizing an inflatable device with an injection pressurizer, the injection pressurizer comprises a housing enclosing a first cavity and a plunger adapted to fit inside said housing,
characterized in that the method comprises:
engaging said plunger into said housing
attaching the injection pressurizer to the inflatable device,
sealing a first volume in a tube cavity in an elongated tube provided in the plunger;
pressing said plunger into said housing such that a pressure indicating piston adapted to be moveably arranged inside said tube cavity and delimiting said first volume in said tube cavity from a second volume, indicates a desired pressure built up in the second volume when the piston is moved by the pressure to equalize the pressures of said first volume and said second volume.

14. Method according to claim 13, comprising twisting the plunger to lock the plunger to the housing.

15. Method according to claim 14, comprising twisting the plunger to unlock the plunger from the housing.

16. Injection pressurizer according to claim 2, wherein said end part comprises a plunger cap which is adapted to seal said first volume.

17. Injection pressurizer according to claim 8, wherein said locking mechanism is adapted to lock respectively release the plunger when the plunger is twisted.
